(19) United States
(12) Patent Application Publication
(10) Pub. No.: US 2026/0354972 A1
(43) Pub. Date: Nov. 12, 2026

(54) SHAPE MEMORY DEVICES AND DELIVERY SYSTEMS

(71) Applicant: Edwards Lifesciences Corporation, Irvine, CA (US)

(72) Inventors: Rani Abdullah Mahmoudi, Huntington Beach, CA (US); Olivia Marie Conroy, New Rochelle, NY (US); Eric Robert Dixon, Villa Park, CA (US); Zachary Charles VanEvery, Lake Forest, CA (US); Kevin M. Golemo, Mission Viejo, CA (US); Mohammad Reza Salari, Irvine, CA (US)

(21) Appl. No.: 19/706,832

(22) Filed: Jun. 12, 2026

Related U.S. Application Data
(63) Continuation of application No. PCT/US2024/060192, filed on Dec. 13, 2024.
(60) Provisional application No. 63/610,875, filed on Dec. 15, 2023.

(51) Int. Cl. A61F 2/24

(52) U.S. Cl. CPC A61F 2/2436; A61F 2/2418; A61F 2210/0028; A61F 2210/0033; A61F 2220/0008

(57) ABSTRACT

Systems, methods and apparatus for transcatheter delivery. The system can be used at a native valve. The system can include a delivery system including a shaft. The system can include a shape memory material. The shape memory material can be disposed at a distal end of the delivery system. The shape memory material can be automatically transitioned from a first predetermined shape to a second predetermined shape in response to a change in temperature. The system can include a heat exchange device in direct contact with the shape memory material. The heat exchange device, when engaged, can cause the shape memory material to transition to the first predetermined shape. The heat exchange device, when the engaged heat exchange device is disengaged, can cause the shape memory material to transition from the first predetermined shape to a second predetermined shape. The system can include a control handle.

### CROSS-REFERENCE TO PRIORITY APPLICATIONS

The present application is a continuation of International Application No. PCT/US2024/060192, filed Dec. 13, 2024, which designates the United States and was published in English by the International Bureau on Jun. 19, 2025 as WO 2025/129111, which claims priority to U.S. Provisional Application No. 63/610,875 filed Dec. 15, 2023, the entire content of which are hereby incorporated herein by reference for all purposes.

### BACKGROUND

In vertebrate animals, the heart is a hollow muscular organ having four pumping chambers: the left and right atria and the left and right ventricles, each provided with its own one-way valve. The natural heart valves are identified as the aortic, mitral (or bicuspid), tricuspid and pulmonary, and each has flexible leaflets that coapt against each other to prevent reverse flow.

Prostheses can be used to correct problems associated with impaired heart valves. For example, mechanical and tissue-based heart valve prostheses can be used to replace impaired native heart valves. More recently, substantial effort has been dedicated to developing replacement heart valves that can be delivered with less trauma to the patient than through open heart surgery. Replacement valves can be designed for percutaneous delivery in so-called transcatheter procedures.

Development of transcatheter heart valves and their delivery systems can be challenging. Obtaining access to perform procedures in the heart or in other anatomical locations may require delivery of devices percutaneously through tortuous vasculature.

In light of the above, a need exists for more advanced transcatheter devices and delivery systems.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

In some implementations, transcatheter devices (e.g., heart valves, implants, replacement devices, repair devices, treatment devices, etc.) herein can incorporate shape-memory effects, including elements capable of multiple shape-memory changes. In some implementations, delivery systems (e.g., transcatheter delivery systems, treatment device delivery systems, repair device delivery systems, implant delivery systems, replacement device delivery systems, heart valve delivery systems, etc.) herein incorporate shape-memory effects, including elements capable of multiple shape-memory changes.

In some implementations, devices herein (e.g., implants, transcatheter heart valves, treatment devices, repair devices, etc.) can include metallic stent features that are trained to assume certain shapes upon heating or cooling the devices to a particular temperature or temperatures. In some implementations, heating a device can be done by direct heating, e.g., heating by electrical stimulation, heating by conduction, heating with fluid that has a higher temperature than the device, etc. In some implementations, cooling a device can be done by direct cooling, such as by conduction, electrical manipulation, using a fluid with a lower temperature than the device, applying cyclic thermal loading under a substantially constant stress field, etc.

In some implementations, a device can comprise extensions or arms (e.g., leaflet capture arms, anchors, annulus anchors, flange(s), ridge(s), etc.) extending from a portion (e.g., an end, a middle, a second end, a side, etc.) of the device. In some implementations, the extensions or arms can be bent, curved, or curled 180° at body temperature, but also can be relatively straightened when cooled.

In some implementations, an internal stent of the device can be trained or shape set (e.g., set to transition to a particular shape as temperature is increased and/or decreased) to radially self-crimp when subjected to a reduced temperature.

In some implementations, various elements within a delivery system can also be trained or shape set so as to enable selective engagement and/or disengagement at different temperatures. In some implementations, various elements within a delivery system can also be trained or shape set so as to enable selective engagement at a temperature (e.g., as a temperature is reached, an element may move into engagement with another element or component). In some implementations, various elements within a delivery system can also be trained or shape set so as to enable disengagement at a temperature (e.g., as a temperature is reached, an element may move out of engagement or disengage with another element or component).

In some implementations, a temperature change can be accomplished through the introduction of a fluid or solution (e.g., saline or other). In some implementations, cooling can be accomplished through the introduction of a cold fluid or cold solution. In some implementations, heating can be accomplished through the introduction of a warm or hot fluid or solution.

In some implementations, a temperature change can be accomplished through the use of a Peltier device (mounted on or in proximity of the convertible element) that can change the temperature of the element until it actuates (e.g., engages, disengages, moves, etc.). In some implementations, cooling can be accomplished through the use of a Peltier device (mounted on or in proximity of the convertible element) that can cool the temperature of the element until it actuates (e.g., engages, disengages, moves, etc.). In some implementations, heating/warming can be accomplished through the use of a Peltier device (mounted on or in proximity of the convertible element) that can warm/heat the temperature of the element until it actuates (e.g., engages, disengages, moves, etc.).

In some implementations, a device (e.g., treatment device, replacement device, repair device, implant, prosthetic heart valve, etc.) disclosed herein for replacing or repairing the function of a defective native heart valve comprises a support structure (e.g., a support stent, a stent, a laser cut body, a woven body, a braided body, etc.) comprising or made from a shape memory material that is adapted to transition to a first predetermined shape/configuration (e.g., a shape set configuration, a configuration the device has been shape set into, a configuration the device has been set to remember, etc.) upon cooling and to a second predetermined shape/configuration upon heating, the second predetermined shape/configuration (e.g., a shape set configuration, a configuration the device has been shape set into, a configuration the device has been set to remember, etc.) being different from the first predetermined shape/configuration.

In some implementations, the device is configured as a heart valve for replacing the function of a defective native heart valve and comprises a support structure configured as a support stent comprising or made from a shape memory material that is adapted to transition to a first predetermined shape/configuration upon cooling and to a second predetermined shape/configuration upon heating. In some implementations, the heart valve further comprises a one-way valve structure mounted within a lumen of the support stent. In some implementations, the one one-way valve structure comprises multiple leaflets (e.g., 2, 3, 4, etc. leaflets) that can open to allow blood flow in a first direction and close to inhibit blood flow in a second direction, the second direction different from the first direction. In some implementations, the one-way valve structure can be formed with leaflets made from pericardium.

In some implementations, a support structure (e.g., which can be a component of and/or be usable with any of the devices herein) can include metallic struts forming a plurality of cells. In some implementations, the first predetermined shape/configuration has a reduced diameter (e.g., relative to the second predetermined shape/configuration, relative to a relaxed state, relative to a relaxed room temperature state, relative to an implanted state, etc.). In some implementations, the reduced diameter can help facilitate or make easier loading of the device into a delivery sheath or delivery catheter (e.g., a capsule of a delivery sheath/catheter, a front region of a delivery sheath catheter, etc.). In some implementations, the reduced diameter can be less than 10 mm.

In some implementations, the second predetermined shape/configuration can have an expanded diameter (e.g., relative to the first predetermined shape/configuration, relative to a reduced diameter state, relative to a relaxed state, relative to a relaxed room temperature state, relative to a cooled state, etc.). In some implementations, the expanded diameter can be a size for implantation at a defective native heart valve. In some implementations, the expanded diameter may be at least 30 mm.

In some implementations, the shape memory material can be shape set at multiple predetermined shapes/configurations by applying cyclic thermal loading under a substantially constant stress field.

In some implementations, the second predetermined shape/configuration of the support stent is desirably sized for implantation in a native valve, e.g., an aortic valve, a mitral valve, a tricuspid valve, a pulmonic valve, etc.).

In some implementations, a valve structure can be coupled to the support structure. In some implementations, a valve structure can be sutured to the support structure. In some implementations, a valve structure can be sutured to a cover, fabric, material, component, etc. that is itself connected (e.g., sutured, adhered, fused, etc.) to the support structure.

In some implementations, the device can comprise a fabric skirt disposed around the support structure for enhanced sealing.

In some implementations, the device can have a proximal inflow end and a distal outflow end.

In some implementations, the support structure can comprise one or more (e.g., 2, 3, 4, etc.) radially compressible frames.

In some implementations, the support structure can comprise extensions or arms (e.g., leaflet capture arms, anchoring arms, anchoring extensions, anchors, etc.) extending from one or more of the one or more frames. In some implementations, one or more or each of the arms can be shape-memory trained or set to multiple shapes/configurations.

In some implementations, one or more or each of the arms can be shape-memory trained or set to convert or transition between a first shape/configuration (e.g., a deployed shape/configuration, etc.) and a second shape/configuration (e.g., a delivery shape/configuration, etc.). In some implementations, one or more or each of the arms can be shape-memory trained or set to convert or transition from the first shape/configuration to the second shape/configuration when cooled below a first temperature lower than body temperature. In some implementations, one or more or each of the arms can be shape-memory trained or set to convert or transition from the second shape/configuration to the first shape/configuration when heated to a second temperature (e.g., body temperature, a temperature above body temperature, a temperature near body temperature, etc.).

In some implementations, one or more or each of the arms can be shape-memory trained or set to convert or transition between a first shape/configuration (e.g., a deployed shape/configuration, etc.), wherein the one or more or each of the arms extend radially outward from the support structure and/or the device, and a second shape/configuration (e.g., a delivery shape/configuration, etc.), wherein the arms are radially inward relative to their position in the deployed shape. In some implementations, one or more or each of the arms can be shape-memory trained or set to convert or transition from the first shape/configuration, wherein the one or more or each of the arms extend radially outward from the support structure and/or the device, to the second shape/configuration, wherein the arms are radially inward relative to their position in the deployed shape, when cooled below a first temperature lower than body temperature. In some implementations, one or more or each of the arms can be shape-memory trained or set to convert or transition from the second shape/configuration, wherein the arms are radially inward relative to their position in the deployed shape, to the first shape/configuration, wherein the one or more or each of the arms extend radially outward from the support structure and/or the device, when heated above a second temperature (e.g., body temperature, a temperature above body temperature, a temperature near body temperature, etc.).

In some implementations, the outflow end of the device and/or support structure can be expelled from a delivery sheath first (e.g., before the inflow end). In some implementations, arms/extensions (e.g., leaflet capture arms, anchor arms, anchor extensions, etc.) can extend distally from or relative to the outflow end. In some implementations, the arms/extensions can extend outwardly (e.g., radially outwardly, etc.) from or relative to a surface of the support structure. In some implementations, the arms/extensions can extend in a first direction relative to the outflow end and transition to extend in a second direction relative to the outflow end different from the first direction. In some implementations, the arms/extensions can extend in a first direction relative to an axis of the support structure and transition to extend in a second direction relative to the axis that is different from the first direction.

In some implementations, the arms/extensions can curl, bend, or curve about 180° in a first shape/configuration (e.g., in a deployed shape/configuration, etc.).

In some implementations, the arm/extensions can have a straight shape or a straightened shape (e.g., a shape straighter than in the first shape/configuration, etc.) in a second shape/configuration (e.g., in a delivery shape/configuration, etc.). In some implementations, the arms/extensions can be configured to extend in a distal direction relative to the outflow end or relative to the support structure.

In some implementations, miniature electric coolers may be mounted on each leaflet capture arm to cool the arms below body temperature.

In some implementations, miniature electric heaters may be mounted on each leaflet capture arm to heat the arms to a desired temperature (e.g., to body temperature, to a temperature close to body temperature, to a temperature above body temperature, etc.).

In some implementations, the device comprises one or more frames. In some implementations, the one or more frames are radially compressible. In some implementations, the one or more frames are radially expandable. In some implementations, the one or more frames can comprise and/or be formed with struts comprising and/or made of a shape-memory material. In some implementations, the one or more frames can be trained/set to radially constrict when subjected to reduced temperatures. In some implementations, the one or more radially compressible frames can be trained/set to radially constrict or contract when subjected to a first temperature below body temperature.

In some implementations, the arms/extensions (e.g., leaflet capture arms, anchor arms, anchor extensions, etc.) are integrally formed with at least one of the one or more frames. In some implementations, the one or more frames comprise an inner frame and an outer frame. In some implementations, the arms/extensions (e.g., leaflet capture arms, anchor arms, anchor extensions, etc.) can be integrally formed with the inner frame. In some implementations, the arms/extensions (e.g., leaflet capture arms, anchor arms, anchor extensions, etc.) can be integrally formed with the outer frame.

In some implementations, a delivery system can comprise a control handle. In some implementations, the delivery system can comprise a flexible access sheath or catheter having a lumen. In some implementations, the delivery system can comprise a delivery catheter (which can be in addition to or in place of the flexible access sheath/catheter). In some implementations, the delivery system can comprise and/or be coupled with a device (e.g., a treatment device, a replacement device, a repair device, an implant, a prosthetic heart valve, etc.), which can be the same as or similar to any of the devices herein or other known devices.

In some implementations, the delivery catheter extends distally from the control handle. In some implementations, the delivery catheter has an outer diameter sized to fit through the lumen of the access sheath. In some implementations, the delivery catheter is formed with a lumen extending therethrough. In some implementations, the delivery catheter has a distal capsule at a distal end region of the delivery catheter.

In some implementations, the device comprises a support structure or stent made from or comprising a shape memory material that is adapted to transition to a first predetermined shape/configuration upon cooling and to a second predetermined shape/configuration upon heating.

In some implementations, the device can comprise a one-way valve structure mounted within a lumen of the support structure or stent. In some implementations, the device has a proximal inflow end and a distal outflow end. In some implementations, the device is adapted to be radially collapsed and loaded inflow end first within a distal capsule or distal region of the delivery catheter.

In some implementations, the support structure or stent can have metallic struts made of or comprising a shape-memory material. In some implementations, the support structure or stent is trained or set to radially constrict when subjected to reduced temperatures. In some implementations, the support structure or stent can have one or more frames. In some implementations, the one or more frames are radially compressible. In some implementations, the one or more frames are radially expandable.

In some implementations, the device and/or the support structure can have arms/extensions (e.g., leaflet capture arms, anchor arms, anchor extensions, anchors, etc.) that extend therefrom. In some implementations, the arms/extensions extend radially outwardly relative to the support structure. In some implementations, the arms/extensions extend from an outflow end or outflow end region of one of the one or more frames. In some implementations, the arms/extensions extend from a middle region of one of the one or more frames. In some implementations, the arms/extensions extend from an inflow end or inflow end region of the one or more frames. In some implementations, the arms extensions extend from multiple different regions of the one or more frames. In some implementations, the arms/extensions extend distally relative to one or more of the one or more frames.

In some implementations, the arms/extensions have a curved or curled shape. In some implementations, the arms/extensions have a portion that extends distally, then turns, curves, or curls to extend proximally. In some implementations, the arms/extensions are configured to extend distally from the outflow end and curl around about 180° in the deployed shape.

In some implementations, the arms/extensions can extend radially outwardly, then move radially inwardly, e.g., to capture tissue. In some implementations, the arms/extensions can be configured to extend in a first direction in a first configuration and extend in a second direction (different from the first direction) in a second configuration. A variety of configurations for the frames and arms/extensions and various combinations of the foregoing are possible.

In some implementations, one, some, or all of the arms/extensions are shape-memory trained/set to transition into a first shape/configuration at a first temperature. In some implementations, one, some, or all of the arms/extensions are also shape-memory trained/set to transition into a second shape/configuration at a second temperature, the second shape/configuration being different from the first shape configuration and the second temperature being different from the first temperature.

In some implementations and at various places in this disclosure, when a shape memory material is trained or set to transition to a first preset shape/configuration at a first temperature and trained or set to transition to a second preset shape/configuration at a second temperature, the material, device, process, etc. may be referred to as “two-way” or “two shape” or “multiple shape”, such as a “two-way shape memory material” or “two-shape memory material” or “two-way shape memory device” or “two-way shape memory component”, “multiple-shape memory material”, etc. “Multiple shape” encompasses two way, but is not limited to two way. In some implementations, the material, device, frame, extension, etc. can be moved into other configurations (e.g., by heating and/or cooling), but is trained/set to transition from the other configurations (e.g., intermediate shapes/configurations, a third shape/configuration, a fourth shape/configuration, a shape/configuration caused by an end user, etc.) to the first preset shape/configuration when the first temperature is reached and transition to the second shape/configuration when the second temperature is reached.

In some implementations, one, some, or all of the arms/extensions can be trained/set to transition from a first shape/configuration or deployed shape/configuration, wherein they extend radially outward from or relative to the device to a second shape/configuration or delivery shape/configuration, wherein one, some, or all of the arms/extensions are radially inward relative to the first shape/configuration or deployed shape/configuration. In some implementations, this can be configured to occur when cooled below a certain temperature lower than body temperature.

In some implementations, in the delivery shape/configuration the arms/extensions uncurl to a straightened shape (e.g., relatively straighter than the deployed shape/configuration). In some implementations, the arms/extensions extend in a distal direction in the delivery configuration.

In some implementations, the one or more frames can be formed with struts made of or comprising a shape-memory material. In some implementations, the one or more frames are trained or set to radially constrict or contract when subjected to reduced temperatures, e.g., when subjected to a temperature below body temperature, when subjected to a temperature at least 20 degrees or more below body temperature, etc.).

In some implementations, the arms/extensions can be integrally formed with one of the one or more frames. In some implementations, the one or more frames comprise an inner frame and an outer frame. In some implementations, the arms/extensions are integrally formed with the inner frame. In some implementations, the arms/extensions are integrally formed with the outer frame.

In some implementations, the arms/extensions can be separately formed, but coupled or connected to one of the one or more frames. In some implementations, the one or more frames comprise an inner frame and an outer frame. In some implementations, the arms/extensions are coupled to or connected with the inner frame. In some implementations, the arms/extensions are coupled to or connected with the outer frame.

In some implementations, the delivery system (e.g., any of the delivery systems disclosed herein) can include a plurality of flexible tethers. In some implementations, the plurality of tethers each emerge from a retrieval hub within the distal capsule. In some implementations, the plurality of tethers loop through one or more eyelets on struts at the inflow end of the one or more frames. In some implementations, the flexible tethers are adapted to pull the device into the distal capsule or distal region of the delivery catheter.

In some implementations, a method of loading a device (e.g., treatment device, replacement device, repair device, implant, prosthetic heart valve, etc.) into a delivery system comprises providing or obtaining a delivery system (which can be the same as or similar to any of the delivery systems disclosed herein and can incorporate some or all of the disclosed delivery system features herein). In some implementations, the delivery system has a proximal control handle. In some implementations, the delivery system has a flexible access sheath having a lumen (e.g., access sheath lumen, first lumen, etc.).

In some implementations, the delivery system has a delivery catheter extending distally from or relative to the control handle. In some implementations, the delivery catheter has an outer diameter sized to fit through the lumen of the access sheath. In some implementations, the delivery catheter has a lumen (e.g., delivery catheter lumen, second lumen, etc.) extending therethrough. In some implementations, the delivery catheter has a distal capsule or distal device retention region at a distal end of the delivery catheter.

In some implementations, the method includes providing or obtaining a device (e.g., treatment device, replacement device, repair device, implant, prosthetic heart valve, etc.), which can be the same as or similar to any of the devices disclosed herein and can incorporate some or all of the disclosed device features herein). In some implementations, the device comprises a support structure or stent made from a multiple-shape or two-way shape memory material.

In some implementations, the material is configured, trained, set, etc. to transition to a first preset shape/configuration upon cooling and to a second preset shape/configuration upon heating. In some implementations, the material is configured, trained, set, etc. to transition to a first preset shape/configuration upon cooling to a first temperature and to a second preset shape/configuration upon heating to a second temperature different from the first temperature.

In some implementations, the device can optionally include a one-way valve structure mounted within a lumen of the support structure or stent. In some implementations, the device has a proximal inflow end and a distal outflow end.

In some implementations, the device is configured such that it can be radially collapsed and loaded inflow end first within the distal capsule or distal retention region.

In some implementations, the method includes positioning the proximal end (e.g., inflow end, etc.) of the device adjacent the distal end of the distal capsule or distal retention region.

In some implementations, the support structure or stent of the device is in a first shape/configuration (e.g., a deployed configuration, an expanded configuration, etc.) when positioning the proximal end of the device adjacent the distal end of the distal capsule or distal retention region.

In some implementations, the method includes cooling the device to cause the support structure to radially constrict or contract. In some implementations, the method includes cooling the device to cause the support structure to radially constrict or contract the support structure or stent to a second shape/configuration (e.g., a delivery configuration, a loading configuration, a collapsed configuration, a constricted configuration, etc.).

In some implementations, the device includes arms/extensions (which can be the same as or similar to any arms/extensions disclosed anywhere herein). In some implementations, the arms extend in a first direction in a first shape/configuration (e.g., a deployed configuration, an expanded configuration, etc.). In some implementations, in the first shape/configuration, the arms/extensions extend radially outward (or in a radially outward direction) relative to an outer wall of the support structure. In some implementations, in the first shape/configuration, distal ends of the arms/extensions extend in a proximal direction (e.g., the first direction is a proximal direction). In some implementations, in the first shape/configuration, distal ends of the arms/extensions extend in a distal direction (e.g., the first direction is a distal direction). In some implementations, in the first shape/configuration, the arms/extensions extend in a distal direction, then curve or curl back in a proximal direction.

In some implementations, the method includes cooling the device to cause the arms/extensions to transition from the first shape/configuration to a second shape/configuration (e.g., a delivery configuration, a loading configuration, a collapsed configuration, a constricted configuration, etc.). In some implementations, in the second shape/configuration, the arms/extensions extend in a second direction different from the first direction. In some implementations, in the second shape/configuration, the distal ends of the arms/extensions extend in a second direction different from the first direction. In some implementations, in the second shape/configuration, the arms/extensions have a different shape (e.g., straightened vs. curved, etc.) than in the first shape/configuration.

In some implementations, the method includes cooling the device to cause both (1) support structure to radially constrict or contract, and (2) the arms/extensions to transition to a different shape and/or point in a different direction (e.g., in the second shape/configuration).

In some implementations, the method includes loading the device into the distal capsule or distal retention region of the delivery catheter.

In some implementations, miniature electric coolers can be mounted on the support structure and/or one or more of the arms/extensions. In some implementations, the step of cooling includes energizing the miniature electric coolers.

In some implementations, the step of cooling includes submerging the device in an ice bath or cold saline solution.

In some implementations, the step of cooling includes causing a fluid at a lower temperature than the device to come into contact with or near the device.

In some implementations, the one or more frames can be formed with or comprise struts made of a shape-memory material. In some implementations, the one or more frames can be trained to radially constrict when subjected to reduced temperatures. In some implementations, the step of cooling includes cooling the struts to cause them to radially constrict.

In some implementations, the delivery system can include a plurality of flexible tethers. In some implementations, the tethers emerge from a retrieval hub within the distal capsule or distal capture region. In some implementations, the tethers loop through one or more eyelets on struts of the support structure (e.g., eyelets at an inflow end of the support structure, etc.). In some implementations, the step of loading includes pulling the flexible tethers proximally to pull the device into the distal capsule or distal retention region.

In some implementations, a funnel or other loading device having a narrow end can be fitted within the distal capsule to facilitate loading of the device into the distal capsule or distal retention region.

In some implementations, the delivery system can include an inner tubular member. In some implementations, the inner tubular member is movable within the distal capsule or distal retention region. In some implementations, the inner tubular member includes a retention member on a distal end of the inner tubular member that is movable within the distal capsule or distal retention region.

In some implementations, the retention member has apertures for receiving and capturing proximal struts at a proximal end or inflow end of the one or more frames. In some implementations, the step of loading includes capturing the proximal struts in the apertures and pulling inner tubular member proximally to pull the device into the distal capsule or distal retention region.

In some implementations, the delivery system comprises a proximal control handle. In some implementations, the delivery system comprises a delivery catheter having a capsule (or distal retention region) along a distal end portion. In some implementations, the delivery catheter includes a bending segment located proximal to the capsule. In some implementations, the bending segment has shape changing wires incorporated into a wall thereof.

In some implementations, an expandable device (e.g., treatment device, replacement device, repair device, implant, prosthetic heart valve, etc.) is adapted to be radially collapsed and positioned within the capsule for advancement through vasculature of a subject (e.g., a living subject, a simulation, etc.).

In some implementations, the delivery system can be configured such that electrically energizing the shape changing wires causes deflection of the delivery catheter. In some implementations, the delivery system can be configured such that electrically heating the shape changing wires causes deflection of the delivery catheter.

In some implementations, the delivery system can be configured such that electrically energizing the shape changing wires causes bending of the bending segment of the delivery catheter. In some implementations, the delivery system can be configured such that electrically heating the shape changing wires bending of the bending segment of the delivery catheter.

In some implementations, the shape changing wires are desirably made from Nitinol.

In some implementations, a temperature of the wires is varied to produce desired changes in length.

In some implementations, the delivery system includes at least 4 shape changing wires evenly distributed around the wall of the delivery catheter or around the wall of the bending segment of the delivery catheter.

In some implementations, the delivery system includes an optional nose cone.

In some implementations, the access sheath extends from the control handle to the nose cone. In some implementations, the delivery catheter extends from the control handle to the nose cone. In some implementations, the inner tubular member extends from the control handle to the nose cone.

In some implementations, the control handle has a fluid port in communication with an internal channel through the access sheath. In some implementations, the control handle has a fluid port in communication with an internal channel through the delivery catheter. In some implementations, the control handle has a fluid port in communication with an internal channel through the inner tubular member.

In some implementations, the delivery system (e.g., the access sheath, delivery catheter, etc.) comprises concentric tubes having overlapping ends. In some implementations, the concentric tubes are inside of an access sheath. In some implementations, the concentric tubes are inside of a delivery catheter. The concentric tubes can be used in a variety of types of catheters. In some implementations, a first concentric tube has an engagement pin that snap fits within an axially-oriented retention slot on a second tube. In some implementations, this is defined by a pair of axially-oriented flanking fingers formed of a shape-memory material trained/set to be rigid at body temperature and more flexible when cooled.

In some implementations, the internal channel terminates at the overlapping ends of the tubes. In some implementations, an expandable device adapted to be radially collapsed is positioned within the capsule for advancement through the vasculature.

In some implementations, introduction of cold fluid into the fluid port renders the flanking fingers more flexible to facilitate removal of the engagement pin from the retention slot and disengagement of the tubes.

In some implementations, there can be two engagement pins on diametrically-opposed sides of the first tube and two retention slots on diametrically-opposed sides of the second tube.

In some implementations, the first tube can be larger than the second tube. In some implementations, the engagement pin(s) extends radially inward from the first tube.

In some implementations, the flanking fingers can be spaced apart a distance which is smaller than the diameter of the engagement pin(s). In some implementations, the retention slot(s) can be provided with a generally circular relief hole at its inner end for receiving the engagement pin(s).

In some implementations, a first tube of the concentric tubes has at least one bent tab cantilevered across opening in a side wall thereof that extends into a similarly-shaped opening in the side wall of a second tube. In some implementations, this creates an interference between the two tubes preventing relative axial and rotational displacement therebetween.

In some implementations, the bent tab is formed of a shape-memory material trained/set such that it is martensitic at body temperature and an austenitic transition temperature is set at a temperature above body temperature, such that the bent tab straightens out past the austenitic transition temperature. In some implementations, heating the bent tab straightens the bent tab out of the opening to permit relative axial and rotational displacement of the tubes.

In some implementations, there can be two bent tabs on diametrically-opposed sides of the first tube and two openings on diametrically-opposed sides of the second tube. In some implementations, the first tube is larger than the second tube and the bent tab(s) extends radially inward into the opening(s).

In some implementations, the bent tab may form an S-shape prior to heating.

In some implementations, the austenitic transition temperature may be about 110 °F.

In some implementations, the control handle may have a fluid port in communication with an internal channel through the distal sheath. In some implementations, the internal channel terminates at the overlapping ends of the tubes, wherein introduction of hot fluid into the fluid port heats the bent tab. In some implementations, the system includes electrical contacts positioned adjacent the bent tab, wherein energizing the electrical contacts heats the bent tab.

In some implementations, a sidewall of the capsule or distal retention region can be at least partly formed by Nitinol elements or other shape memory elements trained to shrink upon a change in temperature.

In some implementations, the Nitinol elements (or other shape memory elements) can be in the form of a braided or woven tube. In some implementations, the system includes electrical leads connected to the braided tube such that the braided tube and the capsule constrict with the application of electrical energy or heat to the braided tube.

In some implementations, the braided tube may be trained to shrink upon cooling.

In some implementations, the control handle can have a fluid port in communication with an internal channel that terminates at the capsule. In some implementations, introduction of cold fluid into the fluid port cools the braided tube.

In some implementations, the Nitinol/shape-memory elements are in the form of a one or more helically-wound wires. In some implementations, the system can have electrical leads connected to the helically-wound wires such that the wires and the capsule constrict with the application of electrical energy or heat to the wires.

In some implementations, the wires may be trained to shrink upon cooling. In some implementations, introduction of cold fluid into the fluid port cools the wires.

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

A further understanding of the nature and advantages of the invention will become apparent by reference to the remaining portions of the specification and drawings.

### BRIEF DESCRIPTION OF THE DRAWINGS

Features and advantages of the present invention will become appreciated as the same become better understood with reference to the specification, claims, and appended drawings wherein:

FIG. 1 is a perspective view of a device configured as a transcatheter heart valve that can be modified in accordance with the principles described herein;

FIG. 2 is a perspective view of a two-frame or dual-stent assembly forming a part of the device of FIG. 1;

FIG. 3 is an exploded view of the two-frame assembly of FIG. 2;

FIG. 4 is a cross-sectional view of the two-frame assembly of FIG. 2 having miniature coolers or heaters mounted on the underside of each leaflet capture arm;

FIG. 5 is a perspective view of an exemplary miniature cooler or heater that can be used for the two-frame assembly in FIG. 4;

FIG. 6A is a schematic sectional view of a heart showing a penultimate step in an example mitral valve replacement procedure utilizing a transcatheter valve delivery system;

FIG. 6B is an enlargement of the transcatheter heart valve illustrating the straightening an arm of the device;

FIGS. 7A-7C schematically show a sequence of constricting a transcatheter heart valve using a funnel to fit within a delivery system distal end;

FIGS. 8A-8C schematically show a sequence of constricting a device in stages to fit within a delivery system distal end;

FIGS. 9A-9C schematically show a sequence of constricting a transcatheter heart valve in stages to fit within a delivery system distal end;

FIG. 10 is a broken view of an example delivery system;

FIG. 11A shows overlapping ends of concentric tubes within the delivery system and an engagement structure therebetween that utilizes shape-memory components, and FIG. 11B shows the two tubes after having been disengaged;

FIG. 12A shows overlapping ends of concentric tubes within the delivery system and an engagement structure that utilizes shape-memory components, and FIG. 12B is an enlargement of the engagement structure;

FIG. 12C is a side view of the overlapping tubes of FIG. 12A showing the engagement structure flanked by electrodes, FIG. 12D shows the engagement structure after having been activated for detachment, and FIG. 12E shows the two tubes after having been disengaged;

FIG. 13 is a broken side view of an example delivery system having a proximal control handle and the capacity for bending a distal region;

FIG. 14 is an enlarged view of a bending section within a distal end of the delivery system seen in FIG. 13, FIG. 14A is a cutaway view of the bending section showing multiple shape-memory pull rods therein, and FIG. 14B is a radial sectional view of the bending section;

FIG. 15 is a schematic sectional view of a heart showing a step in an example treatment procedure utilizing a transcatheter delivery system;

FIGS. 16A and 16B are side views of a distal capsule in the transcatheter delivery system of FIG. 15 before and after radial size reduction;

FIG. 17 is a cutaway view of the distal capsule showing one example of a shape-changing structure for constricting the radial cross-section thereof, and FIG. 17A is a schematic representation of an electric circuit used to initiate the constriction;

FIG. 18 is a cutaway view of the distal capsule showing a second example of a shape-changing structure for constricting the radial cross-section thereof, and FIG. 18A is a schematic representation of an electric circuit used to initiate the constriction; and

FIGS. 19A and 19B show a shape changing element that can be incorporated into a delivery system.

### DETAILED DESCRIPTION

The right ventricle and left ventricle are separated from the right atrium and left atrium, respectively, by the tricuspid valve and mitral valve; i.e., the atrioventricular valves. The septal wall extends between the right atrium and left atrium. The present specification and drawings provide aspects and features of the disclosure in the context of several implementations of treatment devices, replacement heart valves, delivery systems, methods, etc. that are configured for use in the vasculature of a subject (e.g., a living subject, a simulation, etc.), such as for treatment (such as replacement, repair, etc.) of natural heart valves in a subject. Valve treatment of the mitral or tricuspid valves is a primary focus of the examples in the present application, but characteristics of the delivery systems described herein may equally be used for other valve locations, and thus the claims should not be constrained to only mitral or tricuspid valve replacement unless expressly limited.

In some implementations, delivery systems described herein can be used for transfemoral percutaneous delivery of a treatment device, such as a replacement mitral valve or a repair device, to a mitral valve to treat patients with moderate to severe mitral regurgitation (or to a tricuspid valve to treat moderate to severe tricuspid regurgitation). In some cases, for safety and/or other reasons, the disclosed devices may be delivered from the atrial side of the atrioventricular valve or atrioventricular valve annulus. For example, a transatrial approach can be made through an atrial wall, which can be accessed, for example, by an incision through the chest. Atrial delivery can also be made intravascularly, such as from a pulmonary vein. The prosthetic valve can be delivered to the right atrium via the inferior or superior vena cava.

In some cases, left atrial delivery can be made via a transeptal approach. In a transeptal approach, an incision can be made in the atrial portion of the septal wall to allow access to the left atrium from the right atrium (the septum can be approached in one or more of a variety of transcatheter approaches, e.g., transjugularly, transfemorally, etc.).

In short, the treatment device can be delivered via transventricular, transatrial, transjugular, and/or transfemoral approaches with small or minimal modifications to the delivery process. Any of the approaches that travel through any vasculature can be call a transvascular approach as well.

Example transvascular approaches can be found in U.S. Pat. Nos. 10,004,599 and 10,813,757, the entireties of which are hereby incorporated by reference for all purposes, but again, a variety of approaches are possible.

Delivery systems can involve numerous actuators and components that cooperate to assist with navigation and deployment of a medical implant in the body. Delivery systems can be complex mechanical devices, which can be expensive to manufacture and challenging to operate. As such, there is a need for simplified and less complex delivery systems that are capable of achieving the desired purpose. The following describes various solutions that address these needs.

In some implementations, convertible elements of devices and/or delivery systems, which utilize shape-memory materials, such as Nitinol (NiTi), are disclosed herein. Shape memory materials are capable of returning to a previously defined shape or size when subjected to an appropriate thermal treatment. The bases for this behavior are substantially reversible phase changes that occur when the temperature of the material moves below and above its transition (or transformation) temperatures. For instance, a cold sample of the material is deformed so that it changes its shape. However, after heating above a certain characteristic temperature, the sample “remembers” its original shape and spontaneously returns to it. When subjected to mechanical stress, such materials also exhibit other attractive properties such as superelasticity (a.k.a., pseudoplasticity). Depending on the temperature, the material can exist in several forms of internal arrangement.

Nitinol has three stable temperature dependent microstructure phases, which are austenite, R-phase, and martensite. In the nomenclature, Rs, Rf, Ms, Mf, Rs', Rf', As, Af denote the start or finish temperatures for the specific transformation change, where “s” is start and “f” is finish. The specific temperatures at which the transformations occur is dependent on multiple factors including the chemical composition of the material (e.g., the ratio of nickel to titanium, impurities, etc . . . ), and how the material was heat treated. In some circumstances, upon heating, the material may bypass the R-phase and go from martensite to austenite.

NiTi alloys change from austenite to martensite upon cooling from a temperature below a starting temperature Ms. Mf is the final temperature at which the transition to martensite completes upon cooling. Likewise, during heating As and Af are the temperatures at which the transformation from martensite to austenite starts and finishes. U.S. Pat. Nos. 6,837,901 and 7,524,329 provide additional background information about shape-memory materials and are incorporated herein by reference.

Memory effects in medical devices have typically only been one-way, such that a spontaneous change from one configuration to another occurs only at a single temperature (e.g., upon heating. (To obtain a second configuration at a temperature below a transition temperature, it is generally necessary to apply stress.) However, as indicated herein, it is possible to obtain a two-way shape-memory effect, in which a shape-memory material changes to a first predetermined shape/configuration upon cooling and then to a second predetermined shape/configuration upon heating.

Multiple shape memory effects (often described herein for illustration as two-way shape-memory effects) involve a change in shape at multiple transition temperatures. For example, a multiple shape memory effect that is a two-way shape-memory effect can involve a change in shape during cooling below a second transition temperature as well as during heating above a first transition temperature.

The material can remember multiple (e.g., two, three, etc.) different shapes. For example, the material can remember or be set to assume one shape at low temperatures, and one at the high temperature. A material that shows a shape-memory effect during both heating and cooling is said to have two-way shape-memory. The reason the material behaves so differently in these situations lies in training or setting. Training implies that a shape-memory can “learn” to behave in a certain way. Under normal circumstances, a shape-memory alloy “remembers” its low-temperature shape, but upon heating to recover the high-temperature shape, immediately “forgets” the low-temperature shape. However, it can be “trained” to “remember” or to leave some reminders of the deformed low-temperature condition in the high-temperature phases. One way of training the SMA consists in applying a cyclic thermal load under constant stress field.

In one method of obtaining the two-way shape-memory effect, the standard shape setting approach is first done. Second, “training” can be conducted by cooling the material below its Af (so that it's twinned martensite) and then applying a stress to change its shape—applying cyclic thermal loading under a substantially constant stress field. The material is then heated back above its Af so that it's austenitic. At this point, the geometry should be once again back to the original post shape set geometry. The training is repeated as many times as required until the material upon cooling (converted to R-phase or martensite) will transform to the “trained” shape/form without an applied stress.

In both a standard setting of a first shape/configuration and training a second shape/configuration, the predetermined or preset shapes/configurations may adjust somewhat over time, i.e., the shape may drift or alter slightly over time especially with repeated heatings and coolings. Various implementations herein describe a “first shape,” “first configuration,” “second shape,” “second configuration,” “predetermined shape,” “predetermined configuration,” “preset shape,” “preset configuration,” or the like. These shapes/configurations are not required to be 100% rigid and unchanging and can have some minor drift while still being considered the particular shape/configuration. For example, multiple transitions between a curved shape and a straightened shape may lead to the curved shape getting slightly less curved over time or the straightened shape becoming slightly less straightened over time, but these would still be two distinct predetermined or preset shapes and considered the same “first shape,” “second shape,” “predetermined shape,” “preset shape,” etc. as these terms are used.

The convertible element may be both self-expanding and self-contracting. The convertible elements described above that exploit the two-way shape-memory effect may both self-contract and self-expand. In some implementations, a convertible element can be configured to self-expand and/or self-contract. In some implementations, the convertible element may be comprised of more than one shape-memory material. In some implementations, each shape-memory material may have a different transition temperature to return to an initial state. Each shape-memory material may exhibit a one-way shape-memory effect. For example, the convertible element may be comprised in part of a first shape-memory material that expands to an initial state at or above a first transition temperature, and in part of a second shape-memory material that contracts to an initial state at or above a second transition temperature. The first and second shape-memory materials may have different compositions and/or different processing histories.

The convertible elements described herein may be formed from a shape-memory material such as a Ni—Ti alloy (e.g., Nitinol), Cu—Al—Ni alloy, or a Cu—Zn—Al alloy. The shape-memory alloy may exhibit a one-way or two-way shape-memory effect. Preferably, the convertible elements may consist of a Ni—Ti binary alloy. For example, the alloy may include about 51 atomic % of Ni. The balance may be substantially Ti. Additionally, the binary alloy may include very small concentrations of elements other than nickel or titanium, such as, e.g., a few parts per million of copper. Optionally, the convertible elements may be made of a ternary or quaternary Ni—Ti alloy which includes one or more additional alloying elements, such as, for example, Al, Ag, Au, Cu, Fe, Ga, Hf, Ir, Nb, Pd, Pt, Rh, Ta, or W. In some implementations, the convertible elements may include more than one shape-memory alloy.

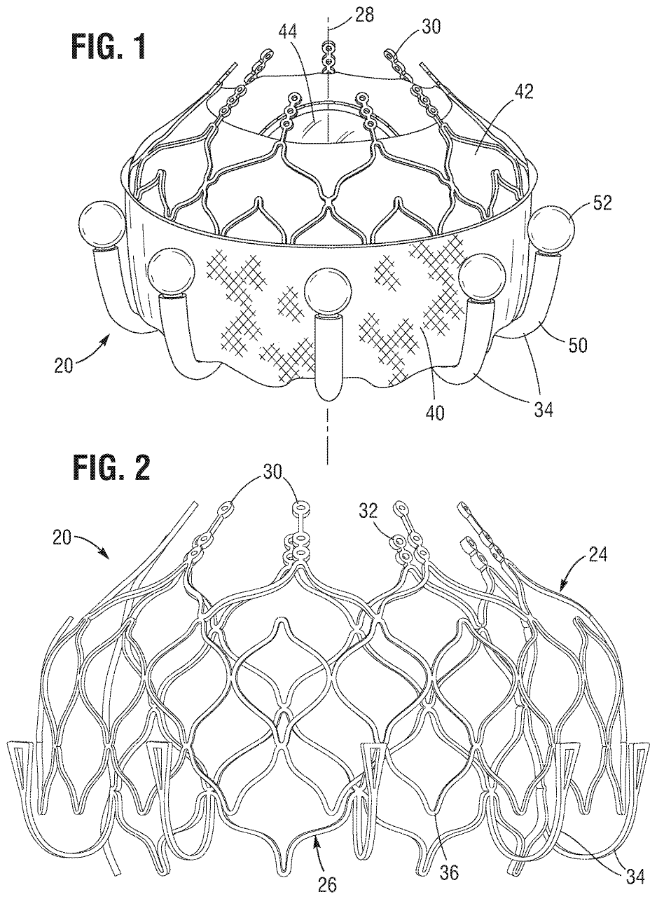

FIG. 1 is a perspective view of a device configured as a transcatheter heart valve 20 that can be modified in accordance with the principles described herein (while a prosthetic heart valve is often used for illustrative purposes herein, it should be understood that similar principles can be applied to other treatment devices (e.g., repair devices, other implants, etc.).

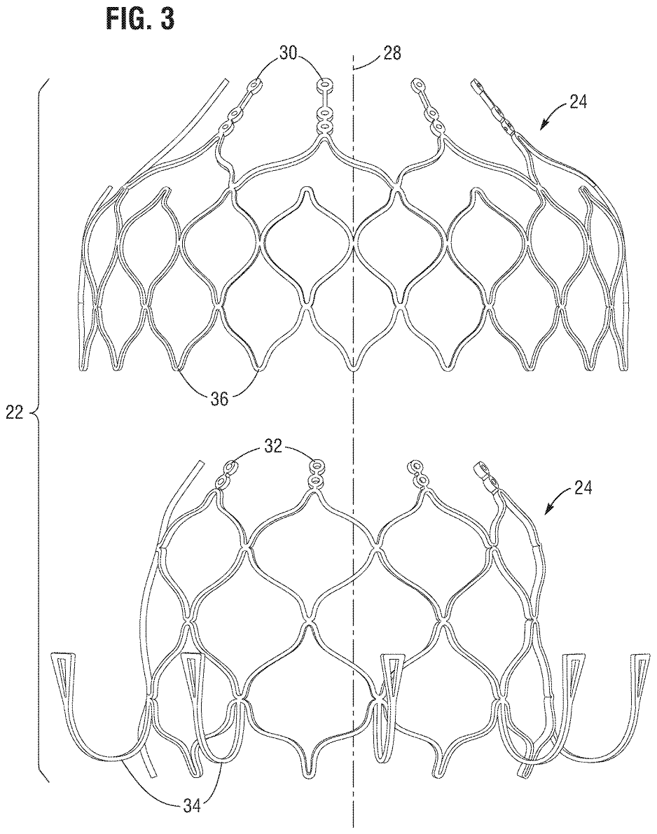

FIG. 2 is a perspective view of a two-frame or dual-stent assembly 22 forming a part of the device, and FIG. 3 is an exploded view thereof. The device or heart valve 20 is shown in an expanded configuration, which occurs at the time of implant. The device or heart valve 20 has an outer stent or frame 24 surrounding and substantially overlapping an inner stent or frame 26, both arranged about a central axis 28. The frames 24, 26 can be made of a biocompatible metal and formed by a plurality of connected struts for contraction and expansion.

In some implementations, as seen in FIG. 2, upper struts 30 of the outer frame 24 extend higher than upper struts 32 of the inner frame 26, while lower leaflet capture arms 34 of the inner frame 26 curl 180° below and radially outward in a deployed shape from a lower end 36 of the outer stent. In some implementations, the arms or extensions 34 are shown or described for illustrative purposes as leaflet capture arms or anchoring arms, but a variety of arms/extensions can be used in a variety of configurations for various purposes (i.e., even if “leaflet capture arms” is used as an example herein, it should be understood that other types of arms/extensions can also be used and in a variety of ways).

In some implementations, arms 34 are in a relaxed configuration when curled this way, a configuration that occurs after coming to body temperature. As will be seen below, although the arms 34 are integrally formed with the inner frame 26, the arms may be trained or set separately to change shape upon cooling in a different manner than the rest of the inner stent. Likewise, the struts of the inner frame 26 other than the arms 34 can be trained or set to change shape in a particular way when cooled.

FIG. 1 shows an outer fabric covering 40 surrounding the outer frame 24, as well as an inner fabric panel 42 within the inner frame 26. The fabric covering 40 and panel 42 are desirably sewn to various points on the respective frames 24, 26, and may be formed from a single sheet or multiple sheets of fabric. A portion of a flexible leaflet 44 is seen through an opening 46 defined by the inner fabric panel 42. A plurality of such leaflets 44 are attached such as by stitches to the fabric panel 42 and surrounding stent structure to form occluding surfaces within an axial flow orifice defined through the valve 20.

Is also seen in FIG. 1, each of the arms 34 of the inner frame 26 has a fabric tube 50 along a majority of its length, and is topped with a rounded fabric bulb 52 at its terminal end. The device or transcatheter heart valve 20 is configured for implantation at one of the atrioventricular valves—the mitral or tricuspid valves. Not all devices described herein need to be implanted, i.e., some can effectuate a treatment and them be removed rather than indefinitely implanted.

In atrioventricular valves, blood flows from the left or right atrium into the left or right ventricle. In FIG. 1, the valve is depicted in a conventional up down orientation wherein blood flow goes downward. Blood depicted or described as flowing downward is generally flowing downstream (even if the heart is not oriented in an up down orientation relative to the ground). An inflow or upstream direction is often depicted and/or referred to as upward and a downflow or downstream direction is often depicted and/or referred to as downward.

In some implementations, arms 34 can be fabric covered. The fabric tube 50 and fabric bulb 52 cover up the otherwise sharp-edged stent arms 34 and protect the heart valve tissue from undue damage.

In some implementations, arms 34 are designed to extend into the ventricle and around the outside of the native heart valve leaflets. The arms 34 thus help anchor the heart valve 20 in place.

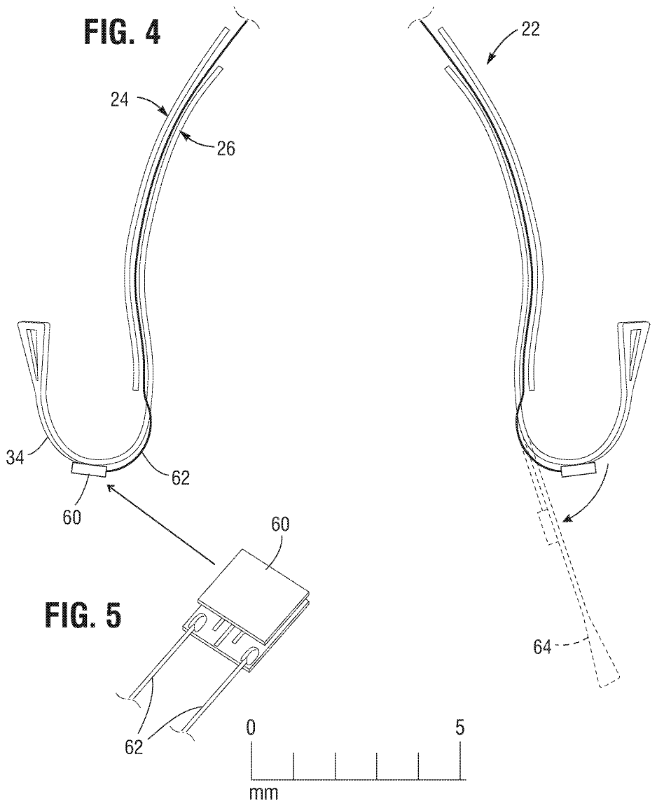

FIG. 4 is a cross-sectional view of the dual-stent assembly 22 modified with miniature Peltier coolers 60 mounted on the underside of each leaflet capture arm 34. The miniature coolers 60 are positioned at a midpoint of the 180° bend in the arms 34, and are supplied with electricity through wires 62 which extend upward between the dual frames 24, 26. Although not shown, the wires 62 can be routed through a delivery system on which is provided an actuator for energizing the coolers 60. Miniature coolers 60 for use in this configuration are available from TEC Microsystems GmbH of Berlin, Germany. Similar miniature heaters that a similarly configured but provide heat can optionally additionally or alternatively be used in similar ways in various implementations herein.

FIG. 5 is a perspective view of an exemplary miniature cooler 60 that can be used for the modified dual-stent assembly 22. The cooler 60 may comprise a Peltier element. A Peltier element is an electrical element which is known from the prior art and which, when subjected to direct current, has a cold side and a warm side. The temperature difference between the warm and the cold side can be controlled, and the cold side and the warm side can be reversed by reversing the polarity. In general, a Peltier element is composed of a number of semiconductor components, also known as p-n junctions, which are mounted between two ceramic panels acting as electrical insulators. If an electric current flows in one direction, heat is conveyed from one side of the semiconductor components to the other side. Reversing the direction of flow/polarity likewise results in a reversal of the conveying direction of the heat. In the present situation, the cold ceramic panel will be mounted against the leaflet capture arm 34. Due to the shape-memory properties of the metal of the leaflet capture arm 34, energizing the cooler 60 cools the arm 34 and causes it to straighten out, as indicated by the dashed outline 64 in FIG. 4.

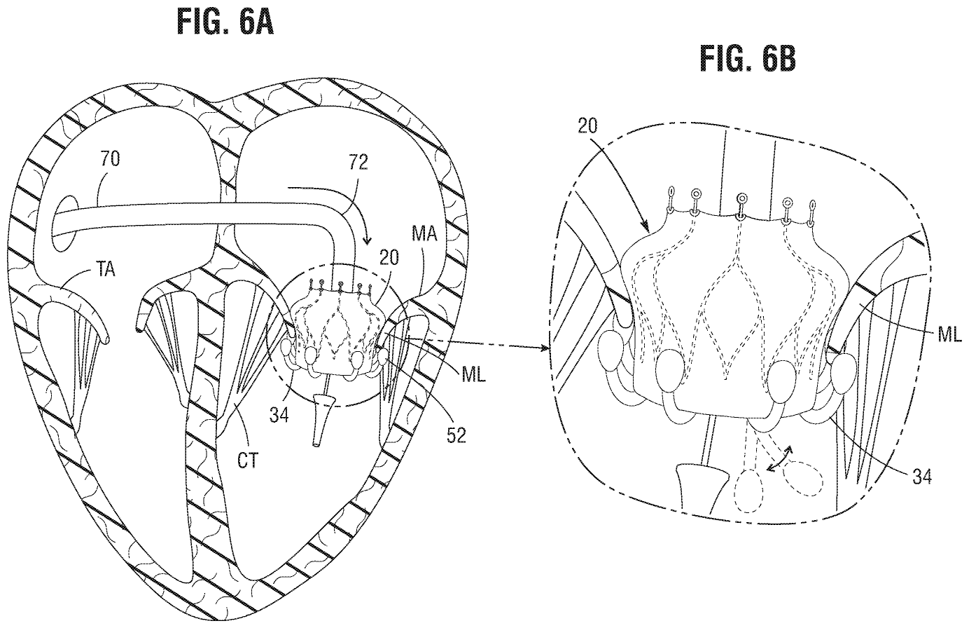

FIG. 6A is a schematic sectional view of a heart showing a penultimate step in an example mitral replacement procedure utilizing a transcatheter valve delivery system, and FIG. 6B is an enlargement of the transcatheter heart valve 20 illustrating the capability of straightening the leaflet capture arms 34. The delivery system includes an elongated sheath or catheter 70, which has a bendable segment 72 adjacent to a distal end thereof. The transcatheter heart valve 20 is crimped or otherwise constricted into a capsule or tube within the delivery system and expelled to expand within the target annulus.

The device 20 in FIG. 6A is shown being implanted at the native mitral annulus MA, though the same could be done at the native tricuspid annulus TA shown on the left side. As mentioned, the leaflet capture arms 34 extend into the ventricle and are designed to curl underneath the mitral leaflets ML. In some implementations, the leaflet capture arms 34 are straightened in a delivery shape within the delivery system and expelled first, whereupon they curl around underneath the mitral leaflets ML. Because of the complicated anatomy within the ventricle, including chordae tendineae CT attached to the mitral leaflets ML, deployment of the leaflet capture arms 34 is sometimes impeded. Consequently, the ability to re-straighten the capture arms 34 by use of the integrated coolers 66 is useful. As seen in FIG. 6B, one of the arms 34 is shown being straightened in the dashed outlines. Under visualization, manipulation of each of the capture arms 34 may be accomplished by energizing selective coolers 60.

With an example Nitinol material, the post shape set frame final austenitic temperature (Af) is approximately +15 C (59 °F). One training regimen includes placing the anchor arms in methanol at −35 C (−31 °F) (converted to twinned martensite) then straightening or unrolling them. The arms are then placed in warm water to convert the material back to an austenitic phase. This was repeated approximately 45 times. The result is that when the arms are cooled to −35 C (−31 °F), they tend to straighten or unroll, but when at body temperature which is above +15 C (59 °F), they return to their shape set curled form. As will be appreciated, a two-way shape-memory effect may be advantageously used to both straighten the arms when needed by cooling and then cause them to bend upon heating, such as to body temperature, thereby providing the clinician with improved control over the deployment of the valve. This also allows one or more arms to be straightened upon demand in the event of misplacement (e.g., not positioned properly behind a native leaflet).

Crimping a dual frame Nitinol stent can have many challenges such as crimping damage, crimping forces, and profile—current mechanical crimping mechanisms can introduce asymmetry in crimping. This invention involves “two-way” shape training the Nitinol frame to be able to “self-crimp.” The frame can be a partially crimped with this technique. Currently, a crimped frame is not cylindrical, but is more like a “cone” which introduces higher crimp strains. The two-way effect with “cooling” could help with crimping a valve cylindrically.

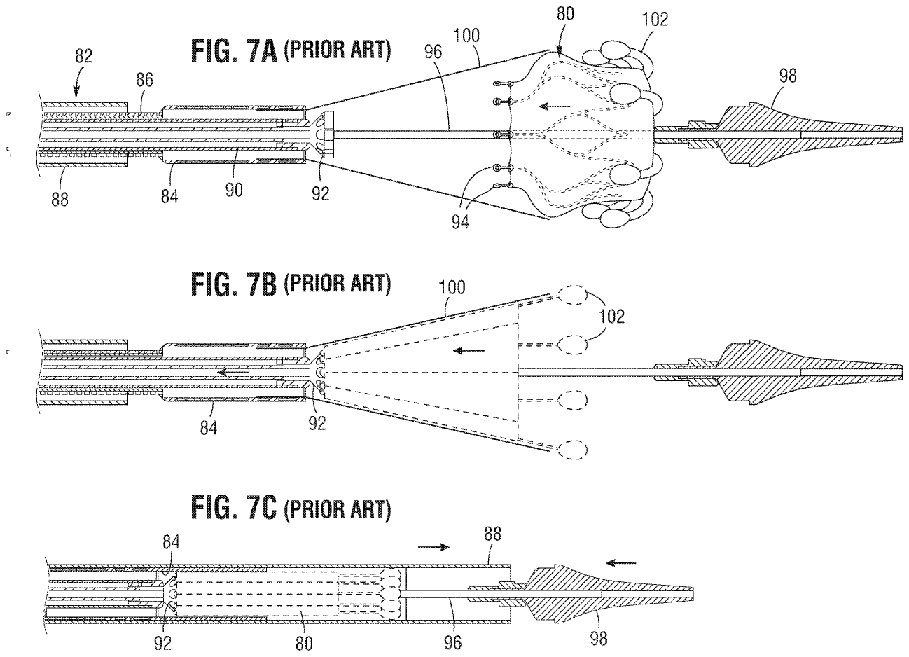

FIGS. 7A-7C schematically show a conventional sequence of constricting a transcatheter heart valve 80 into a valve delivery system 82. While these figures are labeled as “prior art”, some elements, features, steps, etc. described or discussed below with respect to these figures may be inventive, but are merely discussed in the context of otherwise conventional sequencing, i.e., some of the description of or regarding these figures may not be in the prior art.

In some implementations, the valve delivery system may include a distal capsule 84 in tubular form for receiving a proximal end of the crimped heart valve 80. The capsule 84 is shown mounted on the end of a flexible segment 86 of a delivery catheter (not numbered) to facilitate bending and reorientation of the capsule during the final stages of implant. A larger sheath 88 can encompass the entire heart valve 80 during delivery, and is then retracted to expose and permit expansion of the heart valve 80.

In some implementations, the heart valve 80 is packaged and supplied as a separate sterile component from the delivery system, and is coupled thereto at the time of crimping.

In some implementations, an inner tubular member 90 has a conical retention member 92 on its distal end featuring apertures for receiving and capturing proximal struts 94 on the heart valve 80.

In some implementations, a small diameter catheter or tube 96 extends through the tubular member 90 and through the heart valve 166 to be coupled with a nose cone 98 used to facilitate advancement through the vasculature. Although not shown, a guidewire can extend through the tube 96 to also facilitate advancement to the implantation site.

In some implementations, the heart valve 80 is coupled to the delivery system 82 using a temporary funnel 100, having a narrow end fitting closely within the capsule 84. By pushing or otherwise manipulating the heart valve 80 into the funnel 100, the inner self-expanding stent structure is gradually constricted. The leaflet capture arms 102 are straightened out when they contact the wide end of the funnel 100, as seen in FIG. 7B. Eventually, the proximal struts 94 on the heart valve 80 engage the apertures in the conical retention member 92 and are captured thereby. At this point, proximal retraction of the tubular member 90 pulls the heart valve the remaining distance into the delivery system until it reaches the position shown in FIG. 7C. Only a portion of the heart valve 80 is retained within the capsule 84, and the larger sheath 88 is then advanced to the distal direction to contain the entire heart valve. Advancement of the sheath pushes the temporary funnel 100 out of the capsule 84, and it can be removed. The nose cone 98 is also then retracted in a proximal direction to form a seamless connection with the outer sheath 88.

Reducing the diameter of the heart valve 80 would facilitate loading of the into a delivery sheath, but could necessitate high loading forces to crimp the valve stent. Similarly, when straightening the leaflet capture arms 102 out, extremely high bending forces are often required. If this operation is carried out just prior to the valve replacement procedure in the hospital, it can be difficult for even trained technicians. Moreover asymmetry may be introduced in the crimped valve which might affect the valve performance once implanted. Use of materials that are trained for two-way shape-memory so that the crimping can be done in stages can help alleviate these challenges, as described below.

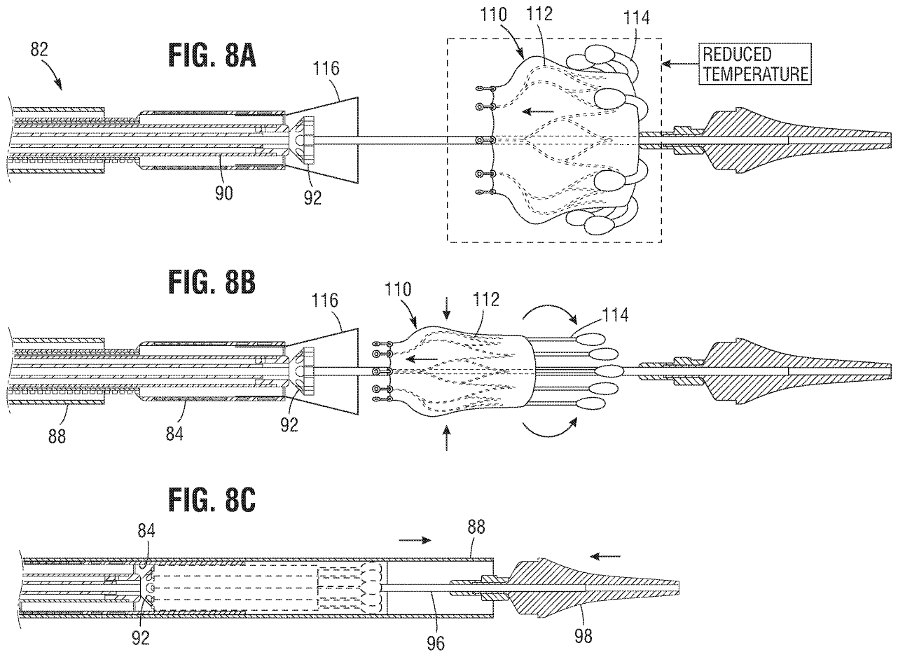

FIGS. 8A-8C schematically show a sequence of constricting a device 110 of the present application into a delivery system 82 in two stages using the two-way shape-memory effect. Components of the delivery system 82 will be given the same numbers as used previously for the sake of consistency, but may have various improvements or differences. In some implementations, a device 110 is held within the delivery system 82 by a retention member 92.

In some implementations, the device or heart valve 110 includes one or more internal stent(s) 112 formed by struts that are made of a material that has been trained to constrict when subjected to reduced temperatures. Likewise, the fabric covered leaflet capture arms 114 are trained to straighten out when cooled. The dashed line box indicating a reduced temperature represents either immersion in a cold saline solution, or cooling using solid elements such as Peltier coolers. The resulting shape change is seen in FIG. 8B. Namely, the internal stent(s) 112 are caused to undergo a reduced diameter, while the same time the leaflet capture arms 114 straighten out.

In some implementations, at the moment that the device or heart valve 110 assumes this intermediate constricted shape, it is caused to enter a truncated temporary funnel 116 whose narrow end is fitted within the tubular capsule 84. Further displacement of the constricted device 110 into the funnel 116 results in the conical retention member 92 engaging the proximal struts on the device. At this point, the device can be pulled into the capsule 84, while the outer sheath 88 is advanced to hold the fully constricted device 110 within the delivery system 82. In short, the two-way shape-memory effect may be advantageously used to assist with crimping by causing the device to automatically reduce in diameter upon cooling, thereby making it possible to pull the device into a capsule or sheath. The reduced diameter of the internal stent(s) 112 facilitates loading of the device into a delivery sheath, and ultimately enables a reduced diameter of less than 10 mm.

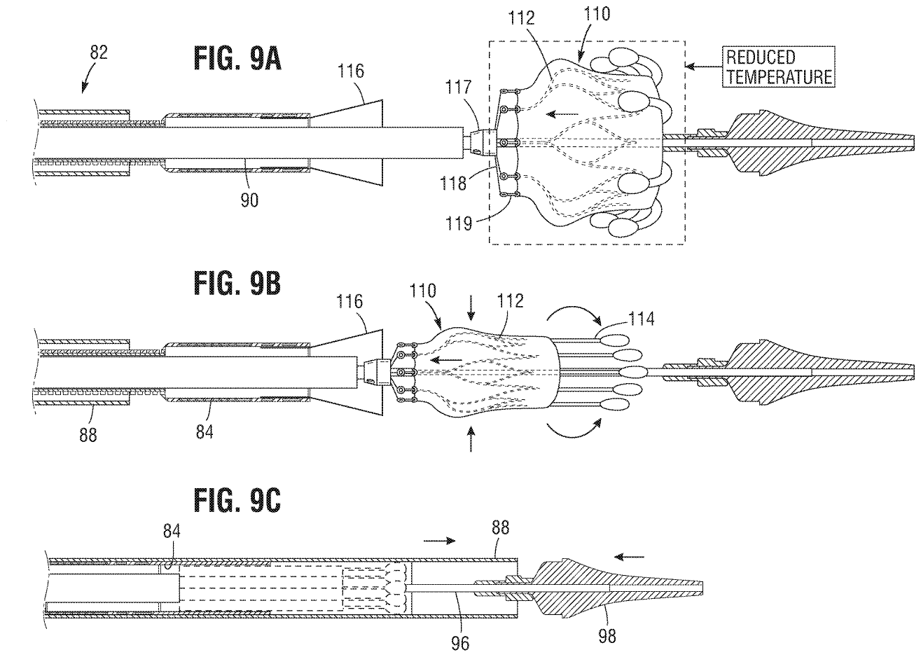

FIGS. 9A-9C schematically show a sequence of constricting the device 110 in stages to fit within the delivery system 82 in a different arrangement. The sequence is essentially the same as described above with respect to FIGS. 8A-8C, but the device or heart valve 110 is pre-coupled to the delivery system 82.

Namely, the inner tubular member 90 within the delivery system 82 houses a shaft at the end of which is a retrieval hub 117. A plurality of flexible tethers 118, such as sutures, extend through the delivery system and emerge from a distal end of the retrieval hub 117. The tethers 118 each loop through an eyelet provided at a proximal end of each of a plurality of atrial stent struts 119 of the device 110. The tethers 118 loop through the eyelets and both free lengths are then threaded proximally back through a passage in the delivery system 82 to a proximal control handle (not shown). The tethers 118 are secured to a movable element within the control handle so the tension can be applied thereto. In this manner, the tethers 118 can be pulled in a proximal direction to cause the eyelets, stent struts 119 and device/valve 110 to be pulled into the capsule 84.

The crimping sequence begins as before by reducing the temperature of the device or heart valve 110, which causes a shape-memory effect of radially constricting the inner stent(s) 112 and straightening the leaflet capture arms 114. Once the heart valve 110 is partially crimped, as shown in FIG. 9B, the tethers 118 are pulled from the proximal control handle to retract the heart valve 110 through the truncated funnel 116 and into the capsule 84. Once again, the outer sheath 88 is advanced and coupled with the nose cone 98, as seen in FIG. 9C.

The devices or heart valves 110 described above include tubular inner stent(s) 112 made from a two-way shape memory material that is adapted to transition to a first predetermined shape/configuration upon cooling and to a second predetermined shape/configuration upon heating. The first predetermined shape/configuration is for loading into the delivery system, while the second predetermined shape/configuration is reached upon implant, when the device comes to body temperature. The inner stent(s) 112 include metallic struts forming a plurality of cells, and the first predetermined shape/configuration has a reduced diameter that may be less than 10 mm. The second predetermined shape/configuration has an expanded diameter sized for implantation within the defective native heart valve, and is preferably at least 30 mm.

FIG. 10 is a broken view of an example delivery system 120 having a proximal control handle 122 from which a distal sheath 124 extends to a nose cone 126. The control handle 122 features a fluid port 128 to which cold saline can be introduced. The fluid port 128 is often used to flush the delivery system 120 of air prior to use. The fluid port 128 can be put in fluid communication via one or more of various internal channels or passages with a variety of structures along the entire delivery system. In this way, cold saline can be delivered to specific locations within the delivery system 120.

The shape-memory effect can be incorporated into various convertible elements within the delivery system 120 itself. For instance, there is often a need to have multiple lumens that can engage and disengage upon demand (such as in the fixation and release of an implant connected to the delivery system). Many current techniques require mechanisms that contribute additional thickness that conflicts with the universal desire to reduce the diameter of delivery catheters.

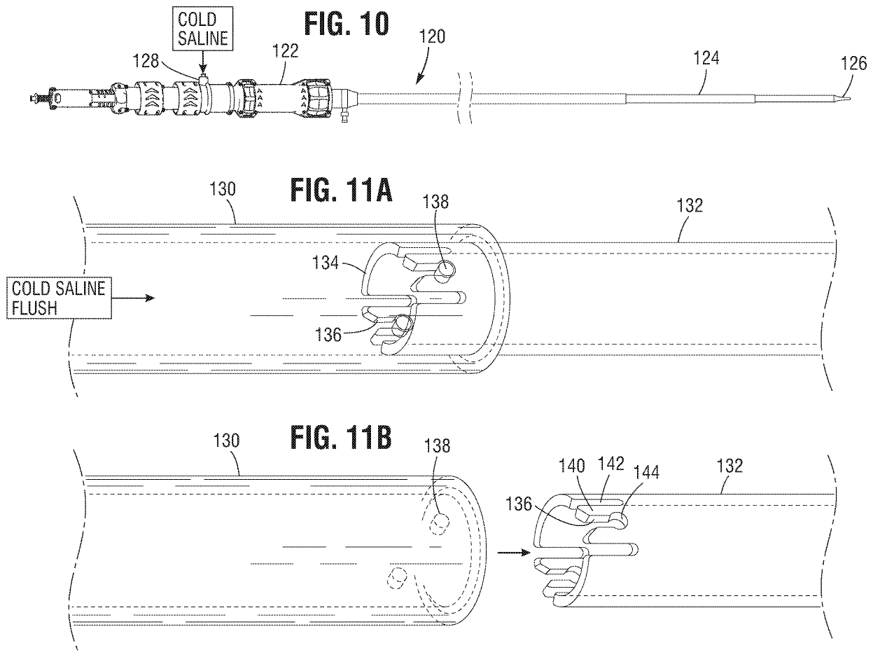

For example, FIG. 11A shows overlapping ends of concentric tubes 130, 132 within the delivery system 120, and an engagement structure therebetween that has two-way shape-memory properties, and FIG. 11B shows the two tubes after having been disengaged. The smaller tube 132 has a terminal end 134 that fits closely within a lumen of the larger tube 130. The terminal end 134 features a pair of axially-oriented retention slots 136 that receive inward pins 138 on the larger tube 130. It should be noted that though the pins 138 are shown on the larger tube 130 and the retention slots 136 on the smaller tube 132, their positions may be reversed.

With reference to FIG. 11B, the retention slots 136 are formed by a pair of axially-oriented flanking fingers 140. The fingers 140 are separated from the remainder of the wall of the smaller tube 132 by a pair of axial relief slots 142. The flanking fingers 140 are spaced apart a distance which is smaller than the diameter of the pins 138, but each retention slot 136 is provided with a generally circular relief hole 144 at its inner end. In addition, the distal ends of each of the fingers 140 are tapered inward such that pushing the two tubes 130, 132 together guides the pins 138 into the slots 136. The pins 138 flex the fingers 140 apart until the pins reach the circular relief holes 144, at which point the fingers 140 are permitted to spring back to their original orientation. This provides a snap-fit between the two tubes 130, 132. Preferably, there are at least two diametrically-opposed retention slots 138 for receiving two similarly positioned pins 138.

The snap-fit features may be designed such that the engagement of the retention slots 138 with the pins 138 on the larger tube 130 will not come undone given the expected relative axial forces the tubes will see procedurally. The smaller tube 132, when designed with a shape-memory effect can be rigid at body temperature. The aforementioned flushing mechanism and fluid port 128 can be used to inject very cold saline solution into the system which will cool the smaller tube 132 to be in a martensitic state, which is more flexible. The fingers 140 can thus be more easily spread farther apart when in the martensitic state, which lowers the force required to disengage the pins 138 and allow for the two tubes 130, 132 to be easily separated.

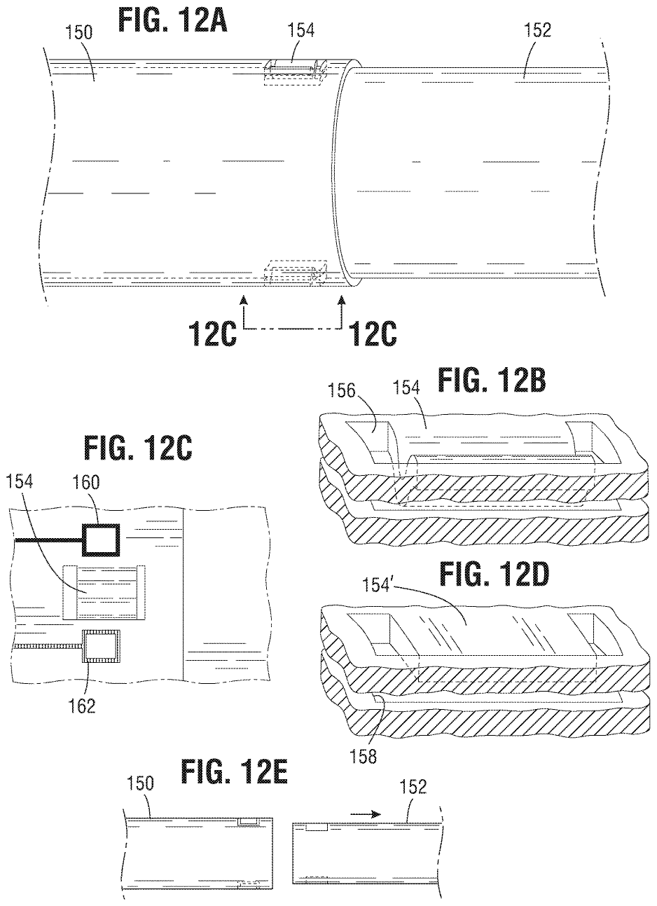

In some implementations, as depicted in FIG. 12A, the delivery system can have overlapping ends of concentric tubes 150, 152 within the delivery system and an engagement structure that has shape-memory properties. The enlargement of FIG. 12B shows that the outer tube 150 has a bent tab 154 cantilevered across at least one opening 156 in a side wall thereof. The tab 154 bends in a generally S-shape radially inward and extends into a similarly-shaped rectangular opening 158 in the side wall of the smaller tube 152. There is thus an interference geometry created between the two tubes 150, 152 preventing relative axial and rotational displacement therebetween. In some implementations, there are two such engagements structures diametrically-opposed across the overlapping ends of the tubes 150, 152. Also, though the bent tab 154 is shown on the larger outer tube 154 and the opening 158 in the side wall of the inner tube 152, their positions may be reversed.

The material of the tab 154 is trained to have shape-memory properties. The composition of the shape-memory material could be such that it is martensitic at body temperature, and the austenitic transition temperature could be set at an elevated temperature (~110 °F). By heating the tab 154, the curved shape can be caused to straighten out as seen in FIG. 12D, thus removing the interference geometry and allowing disengagement of the two tubes 150, 152. FIG. 12E shows the two tubes after having been disengaged. The heating can be achieved by introducing hot saline, such as through the aforementioned flushing system and fluid port 128. Additionally or alternatively, as FIG. 12C illustrates, electrical contacts 160, 162 can be placed adjacent to the area to be heated. Energizing the contacts 160, 162 thus heats the area and causes the bent tab 154 to return to its straight, annealed shape that removes the interference geometry. The tab 154 may be made of a so-called “Muscle Wire.”

Muscle Wire is the name often given to a unique type of wire that acts like the muscles in our bodies. Muscle Wire is a thin wire made from Nitinol (a nickel-titanium alloy) that is known for its ability to contract when an electric current is applied. Made up of equal parts nickel and titanium, Nitinol wire is much stronger than an average strand of metallic wire. The reason Nitinol is able to expand and contract is because of its combination of crystal structures from the nickel and titanium metals. They react differently in high and low temperatures, making the wire soft and flexible when cool, yet firm and stiff when heated.

The means for training Muscle Wire is similar to what is currently done to shape set Nitinol implants. This is a process in which the Nitinol wire/sheet/tube is held into the desired shape (this would correspond to the “activated” current condition), and submerged in a hot bath to “shape set” the metal in the austenitic phase. Lowering the temperature below the Af temperature threshold starts a phase change into the martensitic phase, makes the Nitinol weak, and easily deformable, (this would correspond to the “deactivated” current condition) with no strength in the material. The Af temperature can be manipulated with further exposure to temperature and time during a heat treatment process such that the Af temperature is higher than ambient or body temperature. With no current applied, the wires are weak and in a neutral “deactivated” state, but the application of current through the wire heats them up above the Af temperature into the austenitic phase and the wires remember and transform into the shape they were shape set to.

FIG. 13 is a broken side view of an exemplary transcatheter delivery system 220 having a proximal control handle 222 and the capacity for bending a distal sheath 224 that terminates in a tapered nose cone 226. The particular delivery system 220 and device or heart valve 228 shown is similar to that described above with respect to FIG. 8A. The device or heart valve 228 is shown after expansion, but is delivered in a crimped configuration within the distal sheath 224. Proximal struts on the device/heart valve 228 may be captured by a conical retention member 229 and pulled within a tubular sheath (not shown). In some implementations, a guidewire extends through the tube delivery system to facilitate advancement to the implantation site. The distal sheath 224 features a bending section 230 adjacent to a distal end to enable maneuvering of the distal end for proper positioning of the device/heart valve within the target annulus.

In current delivery systems, bending or flex memory is a major issue. Once the flex is activated, it is extremely difficult to straighten the sheath 224 (one-way flex). Friction in the pull wire mechanisms and plastic deformation of the metal and/or polymer catheter components often mean that the system doesn't have enough elastic strength to return to a straight or nearly straight configuration once tension in the pull wires is removed by reversing the control knobs. This poses many procedural issues, by complicating the maneuverability. Some current delivery systems have 2 planes of flex (primary and secondary) initiated by pull wires controlled by separate “knobs” 232a, 232b on the handle 222, and there can be a lot of cross talk between the two flex planes. The mechanical knobs 232a, 232b activate the flexes and other delivery system mechanics, and require physical input which can approach the ergonomic limit of the physician. For example, in one current system available as the EVOQUE Transcatheter Tricuspid Valve Replacement System from Edwards Lifesciences of Irvine, CA, a primary flex in a distal-most flex section angles the replacement tricuspid valve in the Anterior/Posterior direction (relative to the tricuspid annulus). The EVOQUE system also has a proximal secondary flex section that angles the valve and distal end of the catheters 90° in the septal/lateral direction. These flex and pull wire directions of course can be located along the catheters and oriented to other positions to better navigate toward other valves such as the mitral valve.

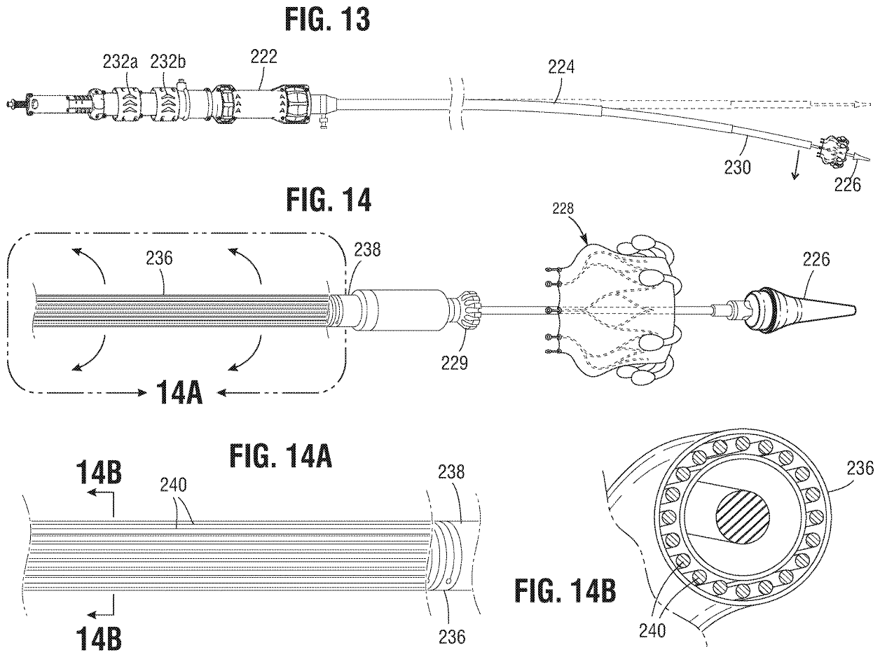

Consequently, FIG. 14 is an enlarged view of a bending section 230 of the present application, and FIG. 14A is a cutaway view of the bending section showing multiple shape-memory pull wires therein. In some implementations, the bending section has a flexible sheath 236 terminating in a solid ring 238. As seen in FIG. 14B, multiple pull wires 240 distributed around the circumference pass longitudinally within sidewalls in the flexible sheath 236 and connect to the ring 238. An independent (or attached) power supply is then hooked up separately to all the Nitinol pull wires 240, and when a charge is applied to heat up certain wires the sheath 236 can flex in multiple planes. Energizing Nitinol tends to heat up the metal which can be trained to cause a shape change, as in Muscle Wire. In this case, shape change is a constriction or shortening of the particular pull wire from heating, which tends to pull the sheath in that direction.

The bending created by the energized pull wires 240 should revert back once electric circuit is deactivated and the system allowed to cool, but to facilitate straightening, opposite wires can be activated. With the use of multiple Nitinol wires 240 as actuators, fine control of the position of the sheath is attained by regulating the amount of electrical current and thus heating that is applied to the various wires. FIG. 14B shows 20 such wires 240 evenly distributed around the sheath 236 which provides very fine control, though as few as 4 and more than 20 may be used. It will be appreciated that incorporating shape changing wires into a delivery catheter can provide significant advantages over conventional pull wires. For example, precise steering can be provided, undesirable cross talk may be eliminated, and actuator knobs may be eliminated from the catheter handle.

In order to flex at separate axial locations at once, there may have to be multiple groups of Nitinol wires at different axial locations independently energized. For example, if it is desirable to only flex the distal end of the catheter, that section may be isolated by shielding the rest of the catheter from reacting to the flexing tension. This can be done through mechanical tension isolating members such as compression coils or pull wire hypotubes around the sections of the wires not intended to flex, or electrical isolation in which dedicated circuits only apply the current to that segment of Nitinol wire. Although not shown, these additional nuances are contemplated.

FIG. 15 is a cross-section of a heart showing a step in an example treatment procedure (e.g., a mitral valve replacement procedure, a mitral repair procedure, a valve replacement procedure, a valve repair procedure, etc.) utilizing a transcatheter delivery system 250. Delivery systems 250 can have a distal portion or capsule 252 that carries the device or heart valve, as well as structures for deploying the device/valve. The capsule 252 is thus often thicker than the rest of the elongated catheter or sheath, which can hinder the ability to maneuver within the vasculature and requires a larger access portal. Consequently, the present application discloses mechanisms for selectively reducing the radial cross-section of the capsule 252.

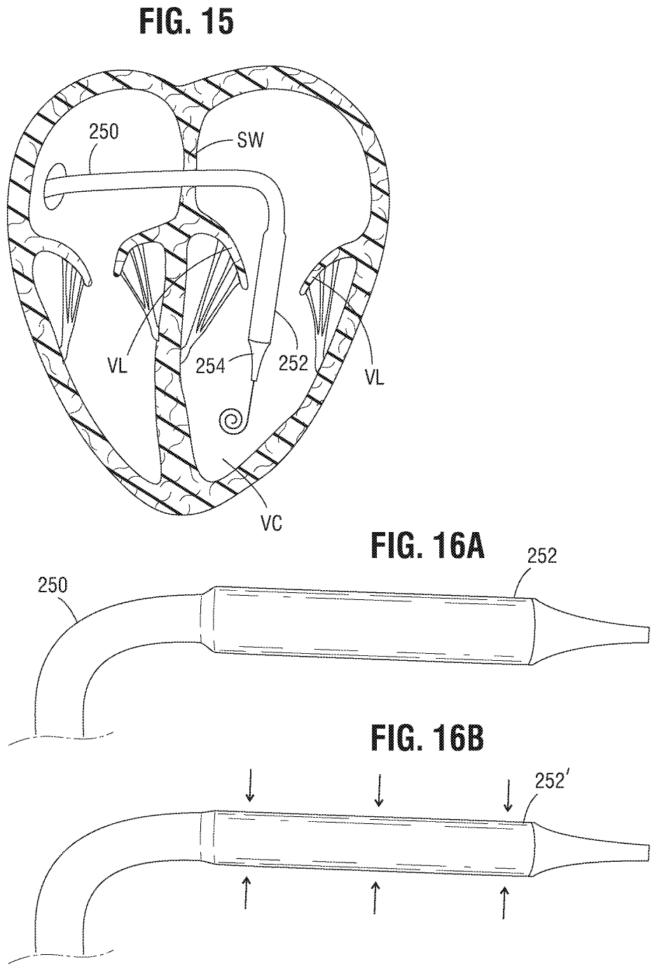

FIGS. 16A and 16B are side views of the distal capsule before 252 and after 252′ radial size reduction. Constructing the sidewall of the capsule 252 of Nitinol in various forms enables the capsule to be shrunk or constricted with the application of energy such as electricity (to generate heat) or heat. Additionally or alternatively, as explained above, the Nitinol can be trained to produce two-way shape changes upon cooling, which can be initiated through the use of a cold flush or with Peltier elements as described.

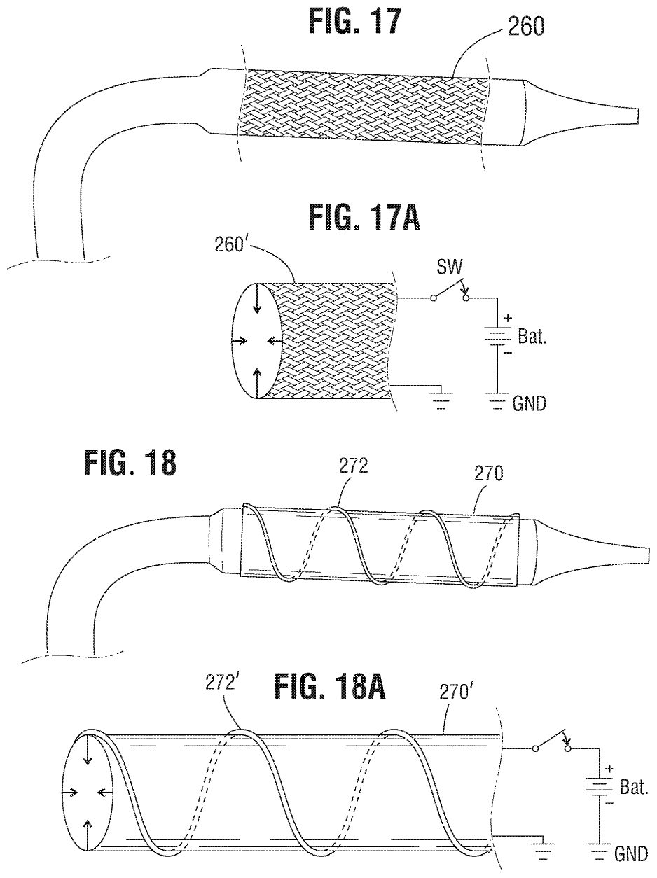

FIG. 17 is a cutaway view of the distal capsule showing one example of a shape-changing structure for constricting the radial cross-section thereof, and FIG. 17A is a schematic representation of an electric circuit used to initiate the constriction. In this configuration, a sidewall of the capsule is formed using a braided Nitinol structure 260. Closing the switch to apply electrical stimulation to the wires within the braided structure 260 causes resistive heating and thus constriction to a changed shape 260′ as seen in FIG. 17A.

FIG. 18 is a cutaway view of the distal capsule showing a second example of a shape-changing structure for constricting the radial cross-section thereof, and FIG. 18A is a schematic representation of an electric circuit used to initiate the heating and constriction. In this case, the outer tubular wall 270 of the capsule is surrounded by one or more helically-wound Nitinol wires 272. FIG. 18A shows the electrical circuit being closed to energize and heat the wire(s) 272 and convert them to a constricted state 272′, thus also constricting the tube 270′. In addition to helically wound wire(s) 272, a laser-cut stent pattern with cell patterns such as lattice, diamond shaped, chevron shaped, or other expanding/contracting stent patterns may be used to constrict the device when energized.

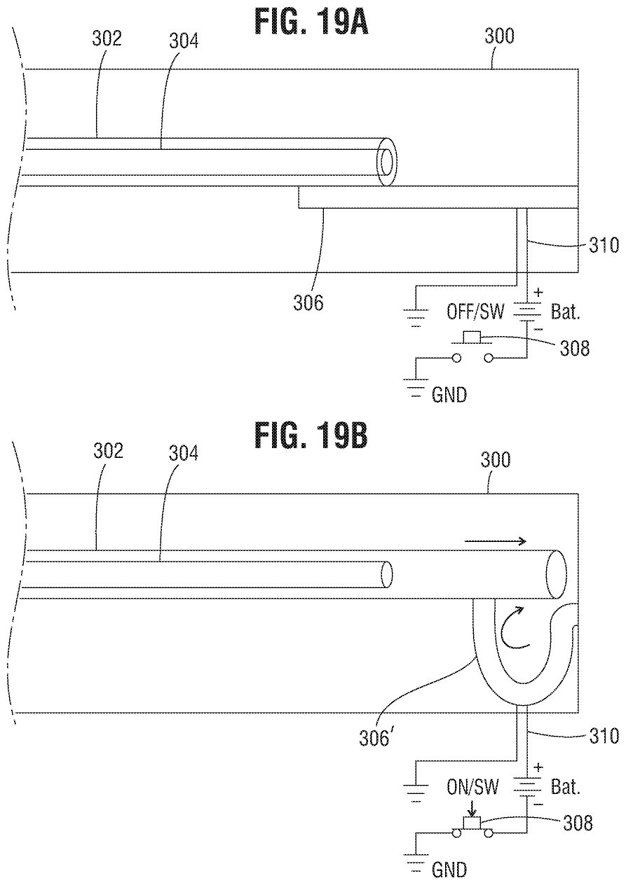

FIGS. 19A and 19B show a shape changing Nitinol (or other shape memory) element that can be incorporated into the delivery system. An outer elongated sheath 300 houses an inner tube 302 within which is positioned a suture 304. Towards a proximal end of the inner tube 302 a trained shape-memory rod 306 attaches. The rod 306 may be energize using a power button 308 and wires 310 to cause the rod to curl into the shape 306′ seen in FIG. 19B. Curling of the rod 306 pulls the tube 302 in a proximal direction which causes release of the suture 302.

This mechanism may be incorporated into systems like FIG. 9A where a device is attached to the delivery system using sutures 118, and can then be released from the delivery system by releasing the sutures. Loops of the sutures 118 are inserted through the inflow eyelets on the atrial struts 119 of the device and doubled back to the delivery system. Although not shown here, a pin (or multiple pins) hold the looped back sutures onto the delivery system. When the pins are retracted, the end of the loop falls off the delivery system and the suture loops are now free to be pulled through the device eyelets thereby permanently decoupling the device and delivery system. The proximal end of this mechanism within the handle has a tube or a rod that gets pulled back a little to allow the loops of suture to slip off it. This retraction of the tube or rod to release the holding pin is what is shown in FIGS. 19A and 19B. There are many various applications for actuating something like a rod or tube a small distance in a handle at the press of a button (sending current through a circuit) rather than rotating a knob, or pulling slider. This could be applied to other mechanisms for other delivery systems, such as a clasp actuator on a device like the PASCAL Precision system from Edwards Lifesciences of Irvine, CA, frame expansion for a valve, release of an implant, flex of a catheter, actuation of a feature or marker visible on echo or fluoroscopy to aid in navigation, etc.

Any of the various systems, assemblies, devices, components, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.).

The techniques, methods, processes, operations, steps, etc. described or suggested herein or in the references incorporated herein, and any methods of using the systems, assemblies, apparatuses, devices, etc. herein, can be performed on a living subject (e.g., human, other animal, etc.) or on a simulation (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

While the foregoing is a complete description of preferred implementations of the invention, various alternatives, modifications, and equivalents may be used. Moreover, it will be obvious that certain other modifications may be practiced within the scope of the appended claims.

What is claimed is:

1. A system for use at a native valve, the system comprising:
a delivery system including a shaft configured to advance an implant to the native valve;
a shape memory material:
having a transition temperature;
configured to be advanced to the native valve; and
disposed at a distal end of the delivery system;
a heat exchange device, in contact with the shape memory material, and operable to cause the shape memory material to contract the implant by changing a temperature of the shape memory material past the transition temperature; and
a control handle that is:
secured to the delivery system; and
configured to operate the heat exchange device.

2. The system according to claim 1, further comprising the implant, the implant including a frame and anchors, the frame and anchors comprising the shape memory material.

3. The system according to claim 2, wherein:
operating the heat exchange device cools the frame and anchors below the transition temperature to constrict the frame and anchors, thereby contracting the implant; and
deenergizing the heat exchange device allows the frame and anchors to warm above the transition temperature, thereby expanding the implant.

4. The system according to claim 2, wherein the heat exchange device includes a Peltier device mounted on one of the anchors.

5. The system according to claim 3, wherein the transition temperature is below body temperature.

6. The system according to claim 2, wherein the frame is expanded and an end of each anchor curves upward and inward above the transition temperature and the frame contracts and the end of each anchor straightens out when the shape memory material is cooled below the transition temperature.

7. The system according to claim 1, wherein the shaft includes a support structure configured to envelope and support the implant, the support structure comprising the shape memory material.

8. The system according to claim 7, wherein:
operating the heat exchange device heats the support structure above the transition temperature to constrict the support structure, thereby contracting the implant; and
deenergizing the heat exchange device allows the support structure to cool below the transition temperature to expand the support structure, thereby allowing the implant to expand.

9. The system according to claim 8, wherein a sidewall of the support structure comprises braided nitinol.

10. The system according to claim 8, wherein:
the support structure includes a tubular capsule and a helically wound wire wound around the capsule; and
the shape memory material comprises the helically wound wire.

11. The system according to claim 7, wherein the heat exchange device includes a Peltier device mounted on an outer surface of the support structure.

12. The system according to claim 7, further comprising a circuit that includes the heat exchange device and the shape memory material;
wherein driving an amount of current through the circuit operates the heat exchange device to cause resistive heating in the shape memory material.

13. The system according to claim 12, further comprising a controller that is configured to select the amount of current in order to control a temperature of the shape memory material.

14. The system according to claim 1, wherein the second predetermined shape of the shape memory material facilitates loading of the shape memory material into the shaft.

15. The system according to claim 1, further comprising a funnel disposed at a distal end of the shaft configured to crimp the implant by pulling the implant into the shaft via the funnel.

16. The system according to claim 15, wherein contracting the implant via the shape memory material contributes to the crimping of the implant.

17. The system according to claim 1, wherein the control handle operates the heat exchange device via application of electrical energy to wires that are secured to the control handle and that extend to the heat exchange device.

18. The system according to claim 1, wherein the shape memory material occupies more volume in the first predetermined shape than in the second predetermined shape.

19. The system according to claim 1, wherein the shape memory material include nitinol.

20. A system for transcatheter delivery of an implant to a native valve, the system comprising:
a delivery system including a support structure at a distal end thereof, the support structure configured to envelope and support the implant, the support structure comprising a shape memory material that:
has a transition temperature;
is configured to be advanced to the native valve; and
contracts the implant when a temperature of the shape memory material is changed past the transition temperature; and
a control handle that is:
secured to the delivery system; and
configured to change the temperature of the shape memory material.
